(19) United States
(12) Patent Application Publication
(10) Pub. No.: US 2014/0081126 A1
(43) Pub. Date: Mar. 20, 2014

(54) APPARATUS FOR GUIDING ENDOSCOPE AND METHOD THEREFOR

(71) Applicant: Electronics and Telecommunications Research Institute, (US)

(72) Inventors: Keo Sik KIM, Gwangju (KR); Hyoung Jun PARK, Gwangju (KR); Kwon Seob LIM, Gwangju (KR); Young Soon HEO, Gwangju (KR); Eun Kyoung JEON, Gwangju (KR); Hyun Seo KANG, Gwangju (KR); Young Sun KIM, Daejeon (KR)

(73) Assignee: Electronics and Telecommunications Research Institute, Daejeon (KR)

(21) Appl. No.: 13/757,889

(22) Filed: Feb. 4, 2013

(30) Foreign Application Priority Data
Sep. 19, 2012 (KR) 10-2012-0104111

(51) Int. Cl. A61B 5/00; A61B 1/04; A61B 1/00; A61B 5/06; A61B 1/07

(52) U.S. Cl. CPC A61B 5/0066; A61B 5/061; A61B 5/0084; A61B 1/07; A61B 1/00165; A61B 1/04; A61B 1/00009; A61B 1/00006; A61B 1/0016
USPC 600/424

(57) ABSTRACT

An apparatus for guiding an endoscope and a method thereof are provided. According to an embodiment of the present invention, there is provided an endoscope guide method including: acquiring tomogram information of tissues based on an optical interference signal according to an optical signal; calculating a distance between an endoscope and the tissues based on the tomogram information; and controlling the location of the endoscope according to the result of comparison between the distance between the endoscope and the tissues and a predetermined reference distance. Accordingly, it is possible to accurately measure the distance between an endoscope and tissues.

### CLAIM FOR PRIORITY

This application claims priority to Korean Patent Application No. 10-2012-0104111 filed on Sep. 19, 2012 in the Korean Intellectual Property Office (KIPO), the entire contents of which are hereby incorporated by reference.

### BACKGROUND

1. Technical Field

Example embodiments of the present invention relate in general to an apparatus and method for guiding an endoscope, and more specifically, to an apparatus and method for guiding an endoscope based on the distance between the endoscope and tissues.

2. Related Art

An endoscopic operation is an operation method that is performed based on images acquired through an endoscope with a camera. The endoscopic operation has been widely performed since it makes tiny incisions and has a high success rate. Conventional endoscopic operations have been performed based on 2D surface images acquired through a 2D camera attached to an endoscope tip. However, the operation method has a problem that tissues may be damaged by a tool for operation since the 2D surface images cannot accurately show the distance between a tool (for example, a scalpel, etc.) for operation and the tissues. Also, due to an operator's vibration or involuntary contraction/relaxation of internal organs, a tool for operation may damage tissues.

In order to overcome the problems, an endoscopic operation method using ultrasound images, an endoscopic operation method using electrical signals, and an endoscopic operation method using a pressure sensor have been used. The endoscopic operation method using ultrasound images has a problem that the distance between a tool for operation and tissues cannot be accurately measured due to the low resolution of ultrasound images. The endoscopic operation method using electrical signals has a problem that accuracy is low and tissues may be damaged due to leakage current since measurement is done by inputting relative values between electrodes to a database. Also, the endoscopic operation method using a pressure sensor has a problem that unnecessary contacts cannot be prevented in advance since a warning signal is transferred after the pressure sensor contacts tissues.

### SUMMARY

Accordingly, example embodiments of the present invention are provided to substantially obviate one or more problems due to limitations and disadvantages of the related art.

An example embodiment of the present invention provides an apparatus for guiding an endoscope, according to the distance between the endoscope (or a tool for operation) and tissues, measured based on optical interferometry.

Another example embodiment of the present invention also provides a method for guiding an endoscope, according to the distance between the endoscope (or a tool for operation) and tissues, measured based on optical interferometry.

In an example embodiment, there is provided an endoscope guide apparatus including: an optical fiber configured to provide a first optical signal to tissues, and to receive a first reflection signal in response to the first optical signal; a reference unit configured to provide a second optical signal to a reference terminal, and to receive a second reflection signal in response to the second optical signal; an optical interferometry unit configured to generate the first optical signal and the second optical signal, and to spectrum-analyze an optical interference signal according to the first reflection signal and the second reflection signal; a location adjusting unit configured to adjust the location of an endoscope; and a processing unit configured to acquire tomogram information of the tissues based on the spectrum-analyzed optical interference signal, to calculate a distance between the endoscope and the tissues based on the tomogram information, and to control the location adjusting unit according to the result of comparison between the distance between the endoscope and the tissues and a predetermined reference distance.

The location adjusting unit may include: an exerciser configured to adjust the location of the endoscope in three axial directions; a motor configured to provide power to the exerciser; and a controller configured to control the operation of the motor according to a location adjustment request from the processing unit.

The processing unit may acquire an A-scan representing the depth of the tissues as the tomogram information.

The processing unit may remove a noise signal from signals included in the tomogram information, change a signal smaller than a predetermined threshold value, among signals which are included in the tomogram information and from which the noise signal has been removed, to the predetermined threshold value, differentiate the resultant tomogram information after changing the signal smaller than the predetermined threshold value to the predetermined threshold value, and calculate an initial inflection point extracted from the differentiated result as the distance between the endoscope and the tissues.

The processing unit may calculate a difference between the distance between the endoscope and the tissues and a predetermined reference distance, and if the difference is smaller than an allowable range, the processing unit may control the location adjusting unit to adjust the location of the endoscope.

In another example embodiment, there is provided an endoscope guide method including: acquiring tomogram information of tissues based on an optical interference signal according to an optical signal; calculating a distance between an endoscope and the tissues based on the tomogram information; and controlling the location of the endoscope according to the result of comparison between the distance between the endoscope and the tissues and a predetermined reference distance.

The acquiring of the tomogram information of the tissues may include acquiring an A-scan representing the depth of the tissues as the tomogram information.

The calculating of the distance between the endoscope and the tissues may include: removing a noise signal from signals included in the tomogram information; changing a signal smaller than a predetermined threshold value, among signals which are included in the tomogram information and from which the noise signal has been removed, to the predetermined threshold value; after changing the signal smaller than the predetermined threshold value to the predetermined threshold value, differentiating the resultant tomogram information; and calculating an initial inflection point extracted from the differentiated result as the distance between the endoscope and the tissues.

The adjusting of the location of the endoscope may include: calculating a difference between the distance between the endoscope and the tissues and a predetermined reference distance; and adjusting the location of the endoscope if the difference is smaller than an allowable range.

Therefore, according to the embodiments of the present invention, by measuring the distance between an endoscope (or a tool for operation) and tissues based on optical interferometry, the distance between the endoscope and the tissues can be accurately measured.

As such, since the distance between an endoscope and tissues can be accurately calculated, it is possible to prevent tissues from being damaged by an endoscope (or a tool for operation).

### BRIEF DESCRIPTION OF DRAWINGS

Example embodiments of the present invention will become more apparent by describing in detail example embodiments of the present invention with reference to the accompanying drawings, in which:

FIG. 1 is a conceptual view showing an endoscope guide apparatus according to an embodiment of the present invention;

FIG. 2 is a block diagram of the endoscope guide apparatus shown in FIG. 1;

FIG. 3 is a flowchart showing an endoscope guide method according to an embodiment of the present invention; and

FIG. 4 is graphs showing results obtained by performing a process of calculating the distance between an endoscope and tissues, according to an embodiment of the present invention.

### DESCRIPTION OF EXAMPLE EMBODIMENTS

Example embodiments of the present invention are disclosed herein. However, specific structural and functional details disclosed herein are merely representative for purposes of describing example embodiments of the present invention, however, example embodiments of the present invention may be embodied in many alternate forms and should not be construed as limited to example embodiments of the present invention set forth herein.

Accordingly, while the invention is susceptible to various modifications and alternative forms, specific embodiments thereof are shown by way of example in the drawings and will herein be described in detail. It should be understood, however, that there is no intent to limit the invention to the particular forms disclosed, but on the contrary, the invention is to cover all modifications, equivalents, and alternatives falling within the spirit and scope of the invention.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element, without departing from the scope of the present invention. As used herein, the term “and/or” includes any and all combinations of one or more of the associated listed items.

It will be understood that when an element is referred to as being “connected” or “coupled” to another element, it can be directly connected or coupled to the other element or intervening elements may be present. In contrast, when an element is referred to as being “directly connected” or “directly coupled” to another element, there are no intervening elements present. Other words used to describe the relationship between elements should be interpreted in a like fashion (i.e., “between” versus “directly between,” “adjacent” versus “directly adjacent,” etc.).

The terminology used herein is for the purpose of describing particular embodiments only and is not intended to be limiting of the invention. As used herein, the singular forms “a,” “an” and “the” are intended to include the plural forms as well, unless the context clearly indicates otherwise. It will be further understood that the terms “comprises,” “comprising,” “includes” and/or “including,” when used herein, specify the presence of stated features, integers, steps, operations, elements, and/or components, but do not preclude the presence or addition of one or more other features, integers, steps, operations, elements, components, and/or groups thereof.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

Hereinafter, embodiments of the present invention will be described in detail with reference to the appended drawings. In the following description, for easy understanding, like numbers refer to like elements throughout the description of the figures, and the same elements will not be described further.

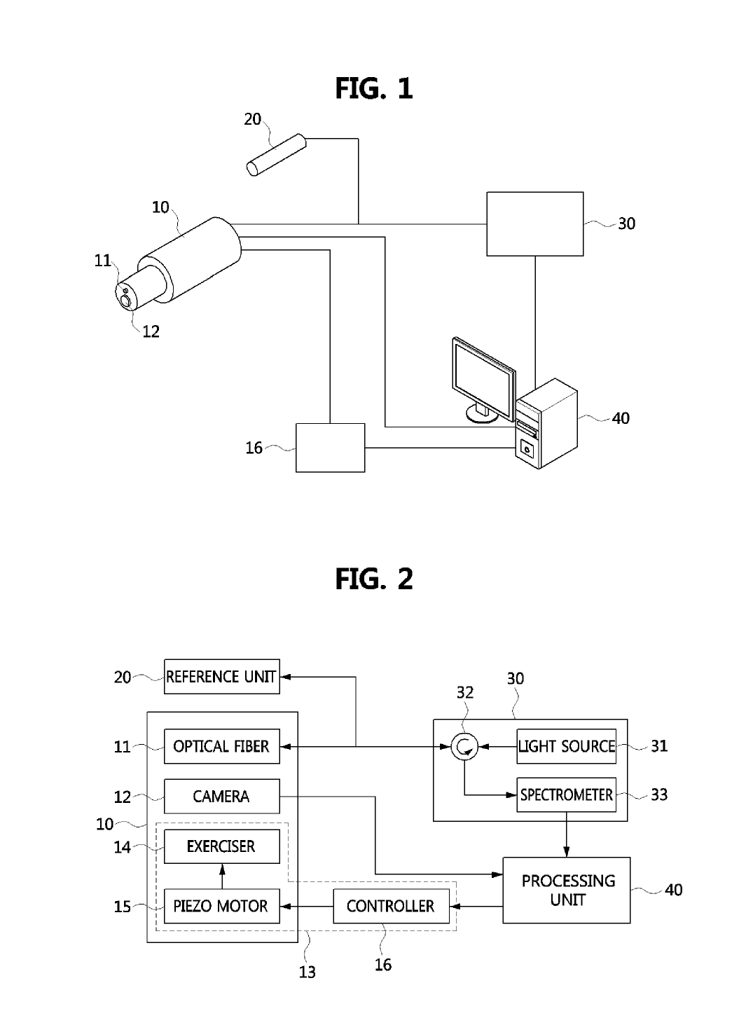

FIG. 1 is a conceptual view showing an endoscope guide apparatus according to an embodiment of the present invention, and FIG. 2 is a block diagram of the endoscope guide apparatus shown in FIG. 1.

Referring to FIGS. 1 and 2, the endoscope guide apparatus may include an optical fiber 11, a location adjusting unit 13, a reference unit 20, an optical interferometry unit 30, and a processing unit 40, and may further include a camera 12.

The optical fiber 11 may provide a first optical signal generated by the optical interferometry unit 30 to tissues (not shown), receive a first reflection signal reflected from the tissues in response to the first optical signal, and provide the first reflection signal to the optical interferometry unit 30. The optical fiber 11 may be positioned in the endoscope 10.

The camera 12 may acquire an image of the tissues, and provide the acquired image to the processing unit 40. The camera 12 may be positioned in the endoscope 10.

The reference unit 20 may provide a second optical signal generated by the optical interferometry unit 30 to a reference terminal (not shown), receive a second reflection signal reflected from the reference terminal in response to the second optical signal, and provide the second reflection signal to the optical interferometry unit 30. The reference unit 20 is a separate element from the endoscope 10, and may be positioned to be separated from the endoscope 10.

Here, the first optical signal and the second optical signal are generated by a light source 31, and may be named differently according to their movement paths. That is, an optical signal that is provided from a circulator 32 to the optical fiber 11 may be referred to as a first optical signal, and an optical signal that is provided from the circulator 32 to the reference unit 20 may be referred to as a second optical signal.

The location adjusting unit 13 may adjust the location of the endoscope 10. The location adjusting unit 13 may adjust the location of the endoscope 10 in three axial directions (X, Y, and Z). The location adjusting unit 13 may include an exerciser 14, a motor 15, and a controller 16. The exerciser 14 and the motor 15 may be located in the endoscope 10, and the controller 16 may be located out of the endoscope 10. Also, the controller 16 may be configured to be included in the endoscope 10 or in the processing unit 40. Here, the motor 15 may be a piezo motor.

The exerciser 14 may convert the rotational motion of the piezo motor 15 to straight line motion, and may include an X-axis exerciser (not shown) for X-axis directional motion, a Y-axis exerciser (not shown) for Y-axis directional motion, and a Z-axis exerciser (not shown) for Z-axis directional motion. The piezo motor 15 may adjust the location of the endoscope 10 in units of “μm,” and may include an X-axis piezo motor (not shown) for the X-axis exerciser, a Y-axis piezo motor (not shown) for the Y-axis exerciser, and a Z-axis piezo motor (not shown) for the Z-axis exerciser.

The X-axis piezo motor may provide power to the X-axis exerciser so that the endoscope 10 moves in the X-axis direction, the Y-axis piezo motor may provide power to the Y-axis exerciser so that the endoscope 10 moves in the Y-axis direction, and the Z-axis piezo motor may provide power to the Z-axis exerciser so that the endoscope 10 moves in the Z-axis direction.

The controller 16 may control the operation of the piezo motor 15 according to a location adjustment request from the processing unit 40. For example, if receiving a request for moving the endoscope 10 in the X-axis direction from the processing unit 40, the controller 16 may operate the X-axis piezo motor, if receiving a request for moving the endoscope 10 in the Y-axis direction from the processing unit 40, the controller 16 may operate the Y-axis piezo motor, and if receiving a request for moving the endoscope 10 in the Z-axis direction from the processing unit 40, the controller 16 may operate the Z-axis piezo motor.

The optical interferometry unit 30 may generate optical signals, and provide the optical signals to the optical fiber 11 and the reference unit 20. Also, the optical interferometry unit 30 may spectrum-analyze an optical interference signal according to the first reflection signal received from the optical fiber 11 and the second reflection signal received from the reference unit 20, and provide the spectrum-analyzed signal to the processing unit 40.

In detail, the optical interferometry unit 30 may include the light source 31, the circulator 32, and a spectrometer 33. The light source 31 may generate optical signals, and the optical signals generated by the light source 31 may be provided to the optical fiber 11 and the reference unit 20 through the circulator 32. The circulator 32 may provide the optical signals generated by the light source 31 to the optical fiber 11 and the reference unit 20, receive the first reflection signal from the optical fiber 11, provide the first reflection signal to the spectrometer 33, receive the second reflection signal from the reference unit 20, and provide the second reflection signal to the spectrometer 33.

Here, the circulator 32 may be replaced with a 2×2 coupler or a 2×1 coupler, and a damper (not shown) may be positioned between the light source 31 and the circulator 32 in order to adjust the intensity of an optical signal generated by the light source 31.

The spectrometer 33 may receive the optical interference signal according to the first reflection signal and the second reflection signal, spectrum-analyze the optical interference signal according to its wavelength to generate a spectrum signal, and provide the spectrum signal to the processing unit 40.

The processing unit 40 may acquire tomogram information of the tissues based on the spectrum-analyzed optical interference signal, calculate the distance between the endoscope 10 and the tissues based on the tomogram information, and control the location adjusting unit 13 according to the results of comparison between the calculated distance between the endoscope 10 and the tissues and a predetermined reference distance.

In detail, the processing unit 40 may represent the optical interference signal according to the first reflection signal received from the optical fiber 11 and the second reflection signal received from the reference unit 20 as equation 1 below.

I(k)=I_{S}(k)+I_{R}(k)+2\sqrt{I_{S}(k)I_{R}(k)}cos [φ_{S}(k)−φ_{R}(k)−kτ], (1)

where I(k) represents the spectrum of the optical interference signal, I_{S}(k) represents the spectrum of the first reflection signal received from the optical fiber 11, I_{R}(k) represents the spectrum of the second reflection signal received from the reference unit 20, Φ_{S}(k) represents the phase of the first reflection signal received from the optical fiber 11, Φ_{R}(k) represents the to phase of the second reflection signal received from the reference unit 20, τ represents fixed optical delay between the first reflection signal and the second reflection signal, and k represents a wave number.

Since the third term of equation 1 includes tomogram information, the processing unit 40 may create equation 2 based on equation 1. That is, equation 1 may be simplified to equation 2, below.

S(k)=2\sqrt{I_{S}(k)I_{R}(k)}cos [φ_{S}(k)−φ_{R}(k)−kτ] (2)

After creating equation 2 based on equation 1, the processing unit 40 may apply an inverse Fourier transform to equation 2, thus creating equation 3 below.

S(z)=F⁻¹{S(k)} (3)

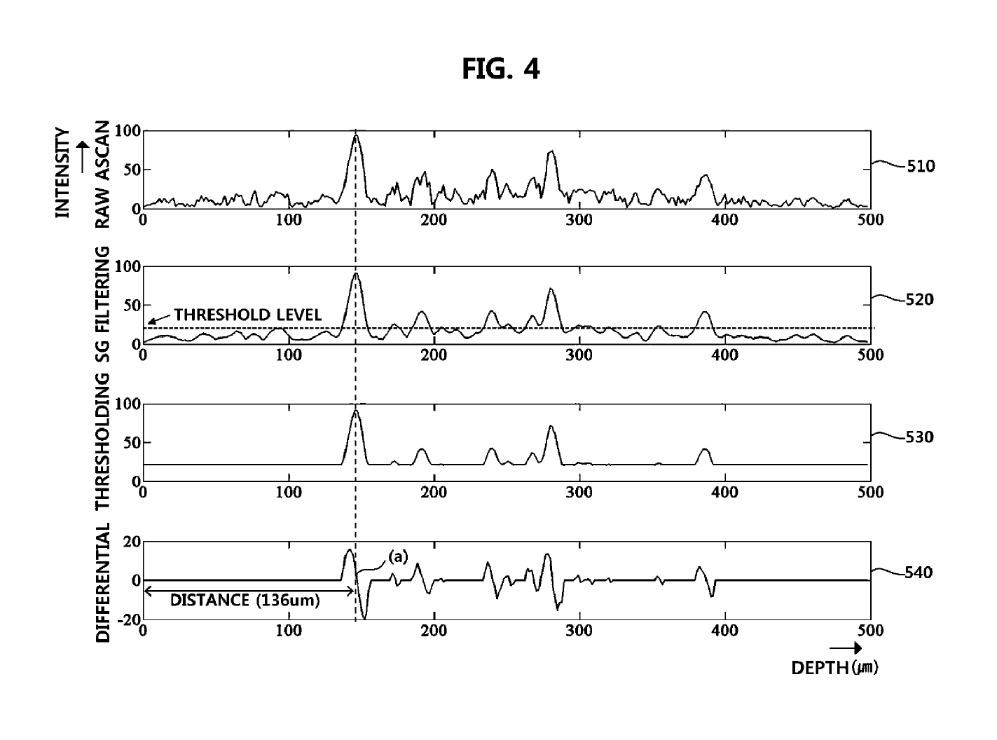

In equation 3, the result S(z) obtained by applying the inverse Fourier transform to equation 2 may be tomogram information, and the processing unit 40 may express the tomogram information as an A-scan. For example, the A-scan of the tomogram information may be represented as a first graph 510 shown in FIG. 4.

FIG. 4 is graphs showing results obtained by performing a process of calculating the distance between an endoscope and tissues, according to an embodiment of the present invention. In FIG. 4, the first graph 510 represents an A-scan of tomogram information, the second graph 520 represents a result obtained by applying a filter to the first graph 510, the third graph 530 represents a result obtained by performing thresholding on the second graph 520, and the fourth graph 540 represents a result obtained by differentiating the third graph 530. In each graph, the X axis represents depth (z), and the Y axis represents intensity (S(z)).

After acquiring the tomogram information, the processing unit 40 may calculate the distance between the endoscope 10 and the tissues. In detail, the processing unit 40 may remove a noise signal from signals included in the tomogram information. At this time, the processing unit 40 may remove a noise signal included in the tomogram information using a filter, and the filter may be a cubic Savitzky-Golay filter (window size: 9 points). However, the dimension of the filter and the window size are not limited to the above example. A Savitzky-Golay filter can minimize the distortion of signals compared to an existing moving average filter or a low pass filter, and does not make signal delay after filtering. The result obtained by applying the filter to the first graph 510 to remove the noise signal, as described above, may be represented as the second graph 520.

The current embodiment relates to the case of removing a noise signal using a Savitzky-Golay filter, however, a filter (preferably a filter making neither signal distortion nor a change in phase) other than the Savitzky-Golay filter may be used. Also, the dimension of the filter and the window size may be set to appropriate values according to a user's purpose.

After removing the noise signal included in the tomogram information, the processing unit 40 may change signals smaller than a predetermined threshold level, among signals included in the tomogram information, to the predetermined threshold level. That is, the processing unit 40 may change signals smaller than a predetermined threshold level to the predetermined threshold level using equation 4 below.

$$S ( z ) = \{ \begin{matrix}S ( z ) , & if S ( z ) \geq threshold \\ threshold , & otherwise\end{matrix} \qquad ( 4 )$$

The predetermined threshold level may be set to an appropriate value according to a user's purpose.

The result obtained by performing thresholding on the second graph 520, as described above, may be represented as the third graph 530, and it is seen from the second graph 520 and the third graph 530 that the predetermined threshold level has been set to about 20.

After changing the signals smaller than the predetermined threshold level to the predetermined threshold level, the processing unit 40 may differentiate the tomogram information, and a result obtained by differentiating the third graph 530 may be represented as the fourth graph 540.

After calculating the differentiated result, the processing unit 40 may extract an initial inflection point from the differentiated result, and calculate the location of the extracted inflection point as the distance between the endoscope 10 and the tissues. That is, in the fourth graph 540, the initial inflection point is (a), and the calculated distance between the endoscope 10 and the tissues is 136 nm.

After calculating the distance between the endoscope 10 and the tissues, the processing unit 40 may control the location adjusting unit 13 according to the result of comparison between the distance between the endoscope 10 and the tissues and a predetermined reference distance.

In detail, the processing unit 40 may calculate the difference between the distance between the endoscope 10 and the tissues and a predetermined reference distance (that is, distance between the endoscope 10 and the tissues—predetermined reference distance), and determine whether the calculated difference is greater or smaller than an allowable range.

If the calculated difference is smaller than the allowable range, the processing unit 40 may adjust the location of the endoscope 10 such that the endoscope 10 moves away from the tissues. That is, the processing unit 40 may request the location adjusting unit 13 to adjust the location of the endoscope 10, and accordingly, the location adjusting unit 13 may control the exerciser 14 and the piezo motor 15 to adjust the location of the endoscope 10 such that the endoscope 10 moves away from the tissues.

Meanwhile, if the calculated distance is greater than the allowable range, the processing unit 40 may maintain the current location of the endoscope 10.

Here, the functions that are performed by the processing unit 40 may be performed substantially by a processor (for example, a CPU (Central Processing Unit) and/or a GPU (Graphics Processing Unit), etc.).

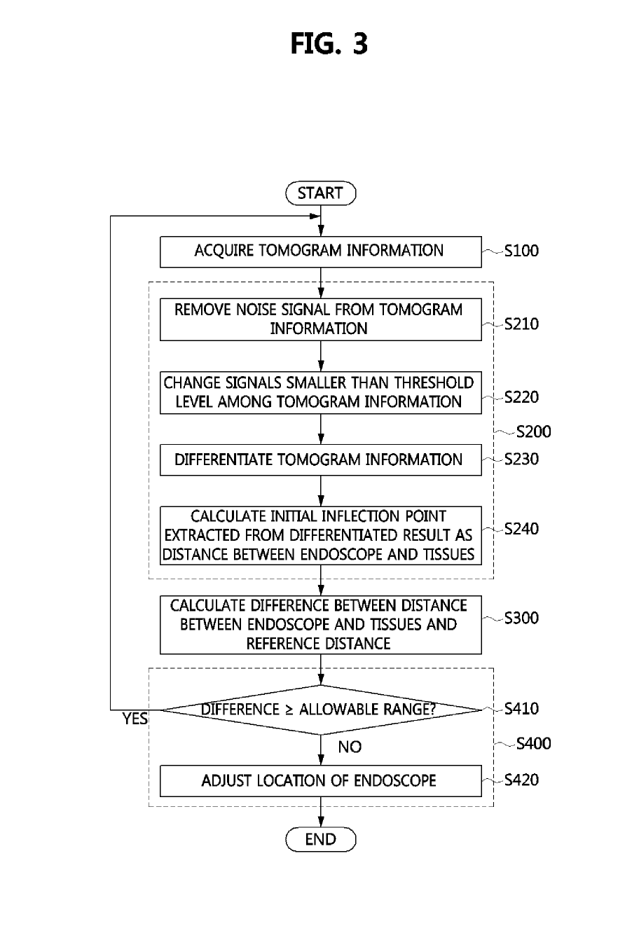

FIG. 3 is a flowchart showing an endoscope guide method according to an embodiment of the present invention.

Referring to FIG. 3, the endoscope guide apparatus (that is, the processing unit 40, see FIG. 2) may acquire tomogram information of tissues based on an optical interference signal according to an optical signal (S 100). That is, the endoscope guide apparatus may represent an optical interference signal according to a first reflection signal from a sample (that is, the tissues) and a second reflection signal from a reference terminal, as the above equation 1, and since the third term of equation 1 includes tomogram information, the endoscope guide apparatus may create the above equation 2 based on equation 1. That is, equation 1 may be simplified to equation 2.

After creating equation 2 based on equation 1, the endoscope guide apparatus may apply an inverse Fourier transform to equation 2, thus creating equation 3.

In equation 3, the result S(z) obtained by applying the inverse Fourier transform to equation 2 may be tomogram information, and the endoscope guide apparatus may express the tomogram information S(z) as an A-scan. For example, the A-scan of the tomogram information may be represented as the first graph 510 shown in FIG. 4.

After acquiring the tomogram information, the endoscope guide apparatus may calculate the distance between the endoscope 10 and the tissues based on the tomogram information (S200). In detail, the endoscope guide apparatus may remove a noise signal from signals included in the tomogram information (S210). At this time, the endoscope guide apparatus may remove the noise signal included in the tomogram information using a filter, and the filter may be a cubic Savitzky-Golay filter (window size: 9 points). However, the dimension of the filter and the window size are not limited to the above example. The result obtained by applying the filter to the first graph 510 to remove the noise signal, as described above, may be represented as the second graph 520.

The current embodiment relates to the case of removing a noise signal using a Savitzky-Golay filter, however, a filter (preferably a filter making neither signal distortion nor a change in phase) other than the Savitzky-Golay filter may be used. Also, the dimension of the filter and the window size may be set to appropriate values according to a user's purpose.

After removing the noise signal included in the tomogram information, the endoscope guide apparatus may change signals smaller than a predetermined threshold level, among signals included in the tomogram information, to the predetermined threshold level (S220). That is, the endoscope guide apparatus may change signals smaller than a predetermined threshold level to the predetermined threshold level using the above equation 4.

The result obtained by performing thresholding on the second graph 520, as described above, may be represented as the third graph 530, and it is seen from the second graph 520 and the third graph 530 that the predetermined threshold level has been set to about 20.

After changing the signals smaller than the predetermined threshold level to the predetermined threshold level, the endoscope guide apparatus may differentiate the tomogram information (S230), and a result obtained by differentiating the third graph 530 may be represented as the fourth graph 540.

After calculating the differentiated result, the endoscope guide apparatus may extract an initial inflection point from the differentiated result, and calculate the location of the extracted inflection point as the distance between the endoscope and the tissues (S240). That is, in the fourth graph 540, the initial inflection point is (a), and the calculated distance between the endoscope and the tissues is 136 nm.

After calculating the distance between the endoscope and the tissues, the endoscope guide apparatus may adjust the location of the endoscope according to the results of comparison between the distance between the endoscope and the tissues and a predetermined reference distance.

In detail, the processing unit 40 (see FIG. 2) may calculate the difference between the to distance between the endoscope and the tissues and a predetermined reference distance (that is, distance between the endoscope and the tissues—predetermined reference distance) (S300), and determine whether the calculated difference is greater or smaller than an allowable range (S410).

If the calculated difference is smaller than the allowable range, the endoscope guide apparatus may adjust the location of the endoscope such that the endoscope moves away from the tissues (S420). That is, the endoscope guide apparatus may request the location adjusting unit 13 (see FIG. 2) to adjust the location of the endoscope, and accordingly, the location adjusting unit 13 may control the exerciser 14 (see FIG. 2) and the piezo motor 15 (see FIG. 2) to adjust the location of the endoscope such that the endoscope moves away from the tissues.

Meanwhile, if the calculated distance is greater than the allowable range, the endoscope guide apparatus may maintain the current location of the endoscope.

The methods according to the present invention may be implemented as program commands that can be executed by various computer means and written to a computer-readable recording medium. The computer-readable recording medium may include a program command, a data file, a data structure, etc. alone or in combination. The program commands written to the medium are designed or configured especially for the present invention, or known to those skilled in computer software. Examples of the computer-readable recording medium include a hardware device configured especially to store and execute a program command, such as a ROM, a RAM, and a flash memory. Examples of a program command include a premium language code executable by a computer using an interpreter as well as a machine language code made by a compiler. The hardware device may be configured to operate as one or more software modules to implement the present invention or vice versa.

While the example embodiments of the present invention and their advantages have been described in detail, it should be understood that various changes, substitutions and alterations may be made herein without departing from the scope of the invention.

What is claimed is:

1. An endoscope guide apparatus comprising:
an optical fiber configured to provide a first optical signal to tissues, and to receive a first reflection signal in response to the first optical signal;
a reference unit configured to provide a second optical signal to a reference terminal, and to receive a second reflection signal in response to the second optical signal;
an optical interferometry unit configured to generate the first optical signal and the second optical signal, and to spectrum-analyze an optical interference signal according to the to first reflection signal and the second reflection signal;
a location adjusting unit configured to adjust the location of an endoscope; and
a processing unit configured to acquire tomogram information of the tissues based on the spectrum-analyzed optical interference signal, to calculate a distance between the endoscope and the tissues based on the tomogram information, and to control the location adjusting unit according to the result of comparison between the distance between the endoscope and the tissues and a predetermined reference distance.

2. The endoscope guide apparatus of claim 1, wherein the location adjusting unit comprises:
an exerciser configured to adjust the location of the endoscope in three axial directions;
a motor configured to provide power to the exerciser; and
a controller configured to control the operation of the motor according to a location adjustment request from the processing unit.

3. The endoscope guide apparatus of claim 1, wherein the processing unit acquires an A-scan representing the depth of the tissues as the tomogram information.

4. The endoscope guide apparatus of claim 1, wherein the processing unit removes a noise signal from signals included in the tomogram information, changes a signal smaller than a predetermined threshold value, among signals which are included in the tomogram information and from which the noise signal has been removed, to the predetermined threshold value, differentiates the resultant tomogram information after changing the signal smaller than the predetermined threshold value to the predetermined threshold value, and calculates an initial inflection point extracted from the differentiated result as the distance between the endoscope and the tissues.

5. The endoscope guide apparatus of claim 1, wherein the processing unit calculates a difference between the distance between the endoscope and the tissues and a predetermined reference distance, and if the difference is smaller than an allowable range, the processing unit controls the location adjusting unit to adjust the location of the endoscope.

6. An endoscope guide method comprising:
acquiring tomogram information of tissues based on an optical interference signal according to an optical signal;
calculating a distance between an endoscope and the tissues based on the tomogram information; and
controlling the location of the endoscope according to the result of comparison between the distance between the endoscope and the tissues and a predetermined reference distance.

7. The endoscope guide method of claim 6, wherein the acquiring of the tomogram information of the tissues comprises acquiring an A-scan representing the depth of the tissues as the tomogram information.

8. The endoscope guide method of claim 6, wherein the calculating of the distance between the endoscope and the tissues comprises:
removing a noise signal from signals included in the tomogram information;
changing a signal smaller than a predetermined threshold value, among signals which are included in the tomogram information and from which the noise signal has been removed, to the predetermined threshold value;
after changing the signal smaller than the predetermined threshold value to the predetermined threshold value, differentiating the resultant tomogram information; and
calculating an initial inflection point extracted from the differentiated result as the distance between the endoscope and the tissues.

9. The endoscope guide method of claim 6, wherein the adjusting of the location of the endoscope comprises:
calculating a difference between the distance between the endoscope and the tissues and a predetermined reference distance; and
adjusting the location of the endoscope if the difference is smaller than an allowable range.
